(19) United States
(12) Patent Application Publication
(10) Pub. No.: US 2026/0148799 A1
(43) Pub. Date: May 28, 2026

(54) APPARATUS AND METHOD FOR PREDICTING THE BINDING STRUCTURE OF A PROTEIN AND A LIGAND

(71) Applicant: Pusan National University Industry-University Cooperation Foundation, Busan (KR)

(72) Inventors: Giltae Song, Yangsan-si (KR); Keumseok Kang, Yangsan-si (KR)

(21) Appl. No.: 19/399,528

(22) Filed: Nov. 24, 2025

(30) Foreign Application Priority Data
Nov. 26, 2024 (KR) 10-2024-0170937

(51) Int. Cl. G16B 15/30; G16B 15/20; G16B 40/20

(52) U.S. Cl. CPC G16B 15/30; G16B 15/20; G16B 40/20

(57) ABSTRACT

Provided are a protein-ligand binding structure prediction apparatus and a method thereof. The apparatus includes: a memory storing a computer program code for predicting the structure of a protein bound to a ligand; and a processor executing the computer program code, wherein the computer program code receives protein information and ligand information, generates a protein vector including distance information between each residue constituting the protein in the protein information, generates a ligand vector that vectorizes each atomic information of the ligand based on the ligand information, generates interaction data including interaction information between each residue and the ligand based on the protein vector and the ligand vector, and predicts the binding structure of the ligand and the protein based on the interaction data.

### BACKGROUND OF THE INVENTION

This invention is related to an apparatus and method for predicting the binding structure of a protein and a ligand.

The description in this section merely provides background information for the embodiments of the present application and does not constitute prior art.

Prediction of residues (or amino acid residues) within a protein involved in ligand interactions provides insights into drug into drug discovery and therapeutics. Broadly speaking, methods for predicting the protein residues binding to a ligand can be categorized into structure-based and sequence-based methods.

Structure-based prediction methods are disadvantageous as the method necessarily requires structural data on proteins and utilization of structural data on large-scale proteins consumes a lot of time and resources.

Due to the disadvantages of the structure-based prediction methods, the sequence-based prediction methods have recently gained attention. However, existing sequence-based prediction methods typically rely on information available only within the protein ligand-binding residues. Accordingly, the existing methods which rely on protein information itself fail to adequately consider interactions with various ligands.

Conventional residue prediction methods utilize several tools to create a feature map when generating a residue representation, and then, analyze the feature map to predict the binding residue. However, protein function can vary depending on the ligand and the site of the protein to which the ligand binds (for example, a pocket), and thus, prediction of ligand-binding residues solely based on protein information will likely suffer from limited accuracies.

Accordingly, a new method is required to predict protein residues binding to a ligand based on both protein information and ligand information.

One of the objectives of the present invention is to provide an apparatus and a method for predicting a protein-ligand binding structure by utilization of both protein information and ligand information.

Further, another objective of the present invention is to provide an apparatus and a method for predicting a protein-ligand binding structure using information of a distance between each residue of a protein. In the present invention, the residue of a protein refers to an amino acid residue of a protein.

The objectives of the present invention are not limited to the stated above. Other objectives and advantages of the present invention not mentioned above can be understood through the following description and will be more clearly understood through the embodiments of the present invention. Furthermore, it will be readily apparent that the objectives and advantages of the present invention can be realized by the means and combinations thereof set forth in the claims.

### SUMMARY OF THE INVENTION

In an embodiment of the present invention, a protein-ligand binding structure prediction apparatus includes: at least one memory storing a computer program code for predicting a structure of the protein capable of being bound to the ligand; and at least one processor configured to execute the computer program code, wherein the computer program code, when executed by the at least one processor, is configured, with the at least one processor, to cause the apparatus at least to: receive protein information and ligand information; generate, based on the protein information, a protein vector including information of a distance between each residue of the protein; generate, based on the ligand information, a ligand vector by vectorizing information of each atom of the ligand; generate interaction data including interaction information between each residue of the protein and the ligand based on the protein vector and the ligand vector; and predict the structure based on the interaction data.

In an embodiment of the present invention, the computer program code can be configured to predict each residue binding to the ligand based on the interaction data.

In an embodiment of the present invention, the computer program code is configured to generate the ligand vector including chemical and physical property information of each atom of the ligand by inputting the ligand information to a graph generation model, and the graph generation model is trained to vectorize the chemical and physical information of each atom of the ligand based on the ligand information.

In an embodiment of the present invention, the computer program code is configured to generate a first protein vector by vectorizing evolutionary information for each residue of the protein based on a protein database.

In an embodiment of the present invention, the computer program code is configured to generate the first protein vector by inputting the protein information to a protein information provision model, and the protein information provision model is trained to generate the first protein vector based on the protein information.

In an embodiment of the present invention, the computer program code is configured to generate, based on the first protein vector, a second protein vector including the information of the distance between each residue.

In an embodiment of the present invention, the computer program code is configured to generate the second protein vector by inputting the first protein vector into a structure inference model, and the structure inference model is trained to: calculate the distance between each residue based on the evolutionary information included in the first protein vector; and generate the second protein vector by adding the information the distance between each residue to the first protein vector.

In an embodiment of the present invention, the computer program code is configured to generate, based on the second protein vector and the ligand vector, the interaction data including the interaction information between each residue and the ligand.

In an embodiment of the present invention, the computer program code is configured to input the interaction data to a binding prediction model so as to identify the residue which binds to the ligand, and the binding prediction model is trained to: calculate a degree of binding between each residue and the ligand based on the interaction data; and determine whether each residue and the ligand are bound to each other based on the calculated degree of binding.

In another embodiment of the present invention, an apparatus for predicting a binding structure between a protein including residues and a ligand is provided. The apparatus includes: at least one memory storing a computer program code for predicting a structure of the protein capable of being bound to the ligand; and at least one processor configured to execute the computer program code, wherein the computer program code, when executed by the at least one processor, is configured, with the at least one processor, to cause the apparatus at least to: receive protein information and ligand information; generate, based on the protein information, a protein vector including coordinate information of each residue of the protein; generate, based on the ligand information, a ligand vector including chemical and physical property information of the ligand; generate, based on the protein vector and the ligand vector, interaction data including interaction information between each residue and the ligand; and predict the structure based on the interaction data.

In an embodiment of the present invention, the computer program code is configured to generate a first protein vector by vectorizing evolutionary information for each residue of the protein based on a protein database.

In an embodiment of the present invention, the computer program code is configured to generate, based on the first protein vector, a second protein vector including coordinate information of each residue in a predetermined space.

In an embodiment of the present invention, the computer program code is configured to generate the second protein vector by inputting the first protein vector into a structure inference model, and wherein the structure inference model is trained to: calculate the coordinate information of each residue in the predetermined space based on the evolutionary information for each residue included in the first protein vector; and generate the second protein vector by adding the coordinate information of each residue to the first protein vector.

In another embodiment of the present invention, a method for predicting a binding structure between a protein including residues and a ligand atoms using a binding structure prediction apparatus is provided. The method includes: receiving protein information and ligand information; generating, based on the protein information, a protein vector including information of a distance between each residue of the protein; generating, based on the ligand information, a ligand vector by vectorizing information of each atom of the ligand; generating, based on the protein vector and the ligand vector, interaction data including interaction information between each residue and the ligand; and predicting the binding structure based on the interaction data.

In an embodiment of the present invention, the generating of the protein vector includes: generating a first protein vector by vectorizing evolutionary information for each residue of the protein based on a protein database; and generating, based on the first protein vector, a second protein vector including the information of the distance between each residue.

In an embodiment of the present invention, the predicting of the structure includes: calculating a degree of binding between each residue and the ligand based on the interaction data; and determining whether each residue and the ligand are bound to each other based on the calculated degree of binding.

In another embodiment of the present invention, a method for predicting a binding structure between a protein including residues and a ligand including atoms using a binding structure prediction apparatus is provided. The method includes: receiving protein information and ligand information; generating, based on the protein information, a protein vector including coordinate information of each residue of the protein in a predetermined space; generating, based on the ligand information, a ligand vector by vectorizing each atomic information; generating, based on the protein vector and the ligand vector, interaction data including interaction information between each residue and the ligand; and predicting the binding structure between the protein and the ligand based on the interaction data.

In an embodiment of the present invention, the generating of the protein vector includes: generating a first protein vector by vectorizing evolutionary information for each residue of the protein based on a protein database; and generating, based on the first protein vector, a second protein vector including the information of the distance between each residue.

In an embodiment of the present invention, the predicting of the structure includes: calculating a degree of binding between each residue and the ligand based on the interaction data; and determining whether each residue and the ligand are bound to each other based on the calculated degree of binding.

In another embodiment of the present invention, a method for predicting a binding structure between a protein including residues and a ligand including atoms using a binding structure prediction apparatus is provided. The method includes: receiving protein information and ligand information; generating, based on the protein information, a protein vector including coordinate information of each residue of the protein in a predetermined space; generating, based on the ligand information, a ligand vector by vectorizing each atomic information; generating, based on the protein vector and the ligand vector, interaction data including interaction information between each residue and the ligand; and predicting the binding structure between the protein and the ligand based on the interaction data.

In an embodiment of the present invention, the generating of the protein vector includes: generating, based on a protein database, a first protein vector by vectorizing evolutionary information for each residue of the protein; and generating, based on the first protein vector, a second protein vector including the coordinate information of each residue.

In another embodiment, a non-transitory computer-readable storage medium storing a program configured to perform the above-stated method.

The protein-ligand binding structure prediction apparatus and the method according to the present invention can increase the accuracy of predicting residues of a protein that bind to a ligand by using protein information, thereby increasing the efficiency in the process of selecting promising candidate substances in virtual screening and the early stages of new drug development. Further, by providing the protein residues that bind the ligand, it is not required to conduct a various number of experiments testing all candidate ligands, which can save time and cost.

Further, by utilizing ligand information, it is possible to predict protein residues bound to new ligands, in addition to protein residues bound to known ligands.

In addition to the above-described, the specific effects of the present invention are described together with the specific matters for carrying out the present invention.

### BRIEF DESCRIPTION OF THE DRAWINGS

FIG. 1 is a block diagram schematically showing the configuration of a protein-ligand binding structure prediction apparatus according to an embodiment of the present invention.

FIG. 2 is a conceptual diagram schematically illustrating an operation of predicting a binding structure of a protein and a ligand by a protein-ligand binding structure prediction apparatus according to an embodiment of the present invention.

FIGS. 3 to 10 are exemplary diagrams for explaining the operation of a protein-ligand binding structure prediction apparatus for prediction of the binding structure of a protein and a ligand according to an embodiment of the present invention.

FIG. 11 is a flowchart illustrating a method for predicting a protein-ligand binding structure according to an embodiment of the present invention.

FIG. 12 is a flowchart illustrating the process of generating the protein vector illustrated in FIG. 11.

FIG. 13 is a flowchart illustrating a process for predicting the binding structure of a ligand and a protein as illustrated in FIG. 11.

FIG. 14 illustrates graphs of binding affinity values measured by a protein-ligand binding structure prediction apparatus according to an embodiment of the present invention and conventional protein structure prediction models.

FIG. 15 illustrates an experimental result of binding affinity values of an active ligand and a decoy ligand measured by a protein-ligand binding structure prediction apparatus according to an embodiment of the present invention.

### DETAILED DESCRIPTION OF THE INVENTION

In the following detailed description, numerous specific details are set forth in order to provide a thorough understanding of embodiments. However, it will be understood by those of ordinary skill in the art that the embodiments can be practiced without these specific details. In other instances, well-known methods, procedures, components and circuits have not been described in detail so as not to obscure the embodiments.

The following description with reference to the accompanying drawing illustrates specific embodiments to enable those skilled in the art to practice them. Other embodiments can incorporate structural, logical, process, and other changes. Portions and features of some embodiments can be included in, or substituted for, those of other embodiments. Embodiments set forth in the claims encompass all available equivalents of those claims. The example embodiments are presented for illustrative purposes only and are not intended to be restrictive or limiting on the scope of the disclosure or the claims presented herein.

The functions described herein can be implemented in software in one embodiment. The software can consist of computer executable instructions stored on computer readable media or computer readable storage devices such as one or more non-transitory memories or other type of hardware-based storage devices, either local or networked.

Although the following description uses terms “first,” “second,” and the like and “A”, “B”, and the like to describe various elements, these elements should not be limited by the terms. The terms are used only to distinguish one element from another. For example, without departing from the scope of the present invention, the first element can be referred to as the second element, and similarly, the second element can also be referred to as the first element.

The terminology used in the description of the embodiments herein is for the purpose of describing a particular embodiment only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof. Throughout the specification, when an element is referred to as being “connected or coupled” to another element, it can be directly connected or coupled to the other element or intervening elements can be present.

The terms or words used in this specification and the claims should not be interpreted as limited to their general or dictionary meanings. In accordance with the principle that the inventor can define the concept of a term or word in order to best explain his or her invention, they should be interpreted as meanings and concepts that are consistent with the technical idea of the present invention. In addition, the embodiments described in this specification and the configurations illustrated in the drawings are only one embodiment in which the present invention is realized, and do not represent the entire technical idea of the present invention, so it should be understood that there can be various equivalents, modifications, and applicable examples that can replace them at the time of this application.

The terminology used in this specification and claims is for the purpose of describing specific embodiments only and is not intended to limit the present invention. The term "and" "or" or "and/or" includes any combination of a plurality of related listed items or any item among a plurality of related listed items. A singular expression includes a plural expression unless the context clearly indicates otherwise. The plural expressions can include a singular expression unless otherwise indicated. It should be understood that the terms "comprise" "include" or "have" in this application do not exclude in advance the possibility of the presence or addition of features, numbers, steps, operations, components, parts or combinations thereof described in the specification.

Unless otherwise defined, all terms used herein, including technical or scientific terms, have the same meaning as commonly understood by one of ordinary skill in the art to which this invention belongs.

Terms defined in commonly used dictionaries should be interpreted as having a meaning consistent with the meaning they have in the context of the relevant technology, and shall not be interpreted in an ideal or overly formal sense unless explicitly defined in this application. In addition, each configuration, process, process, or method included in each embodiment of the present invention can be shared within a scope that is not technically contradictory to one another.

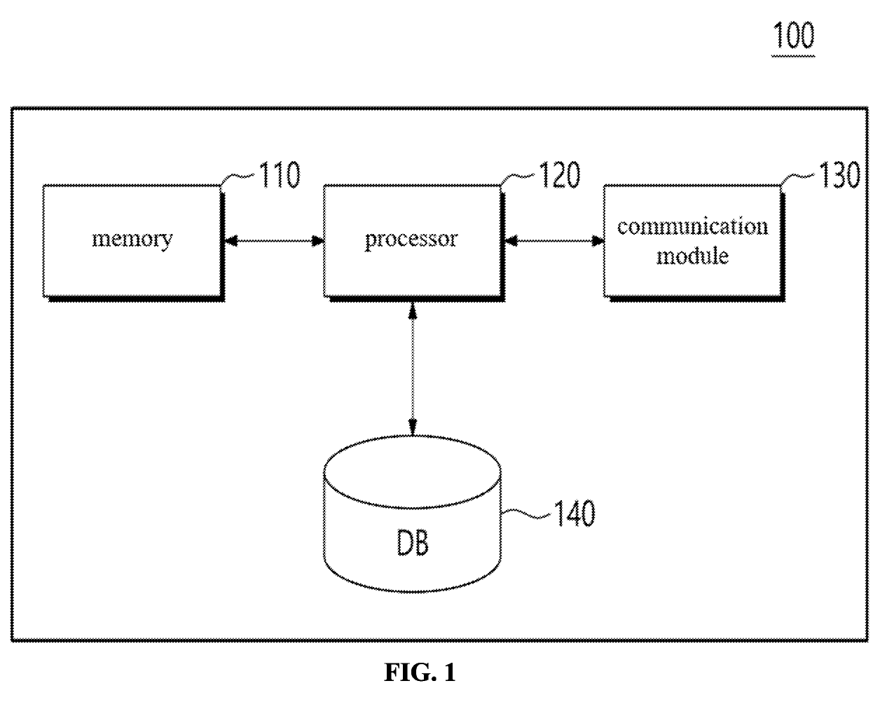

Hereinafter, with reference to FIGS. 1 to 13, an apparatus and method for predicting the binding structure of a protein and a ligand will be described in detail.

First, an apparatus for predicting the binding structure of a protein and a ligand will be described in reference to FIGS. 1 to 10.

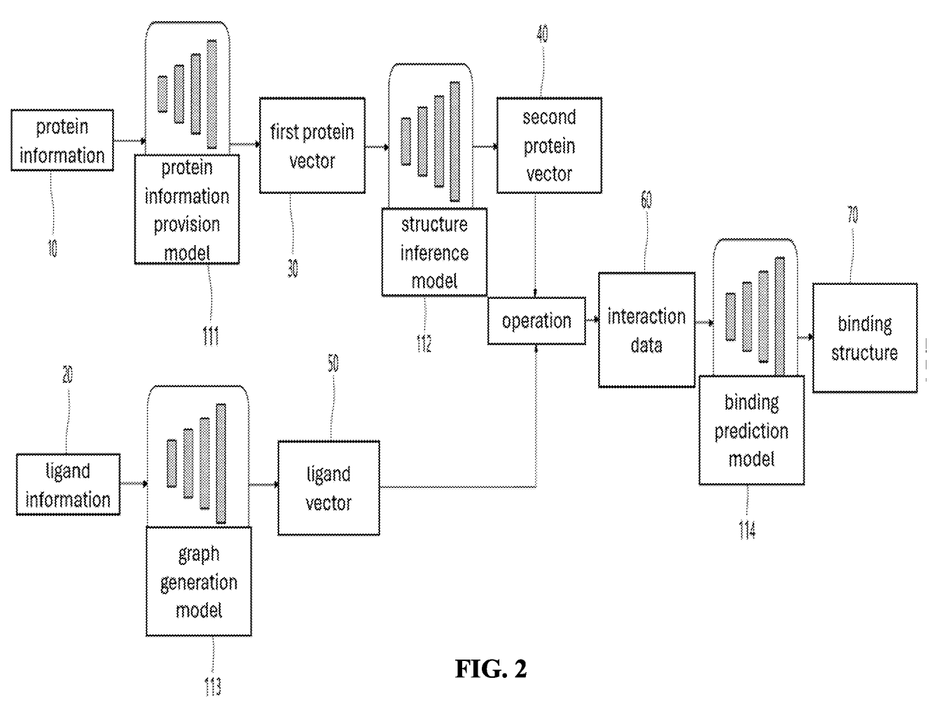

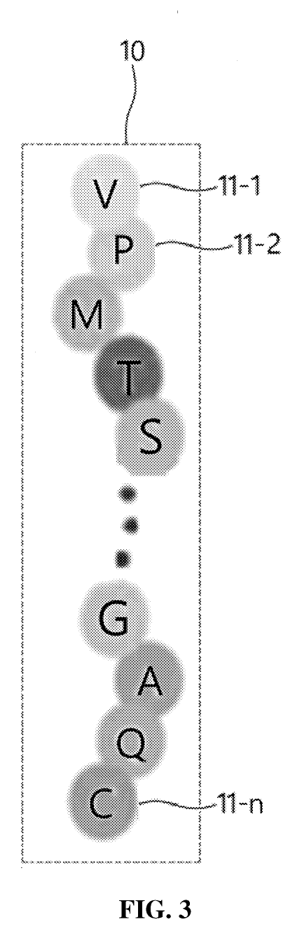

FIG. 1 is a block diagram schematically illustrating the configuration of an apparatus for predicting the binding structure of a protein and a ligand according to an embodiment of the present invention. FIG. 2 is a conceptual diagram schematically showing the operations to be performed by the apparatus for predicting the binding structure of the protein and the ligand to an embodiment of the present invention. FIGS. 3 to 10 are exemplary diagrams illustrating how the apparatus predicts the binding structure of a protein and a ligand, including how the vectors are generated, and the binding structure is imaged in 3 dimensions, according to an embodiment of the present invention.

Referring to FIGS. 1 and 2, a protein-ligand binding structure prediction apparatus (100) receives protein information and ligand information, and can generate a protein vector including information on each residue constituting the protein based on the protein information. The protein information can include information about each amino acid base sequence in the protein sequence. Further, a ligand vector that vectorizes each atomic information of the ligand based on the ligand information can be generated, and interaction data including information on the interaction between the protein and the ligand can be generated through the protein vector and the ligand vector. Furthermore, based on the interaction data, residues of the protein that bind to the ligand can be estimated, and thereby the binding structure of the protein and the ligand can be predicted.

The protein vector includes distance information between each residue or coordinate information of each residue with respect to a given space, and the ligand vector includes chemical and physical property information for each atom of the ligand. Further, the interaction data includes reaction information between each residue of the protein and each atom of the ligand.

In other words, the protein-ligand binding structure prediction apparatus (100) predicts the binding structure between a protein and a ligand based on three-dimensional structural information of the protein using distance information for each residue of the protein or coordinate information for a predetermined space, thereby increasing prediction accuracy. To perform such operations, the protein-ligand binding structure prediction apparatus (100) can include a memory (110) and a processor (120).

The memory (110) can store a prediction program that predicts the binding structure of a protein and a ligand based on received protein information and ligand information. The memory (110) can be interpreted as a general term for both non-volatile storage devices that maintain stored information even when power is not supplied and volatile storage devices that require power to maintain the stored information. Furthermore, the memory (110) can perform a function of temporarily or permanently storing data processed by the processor (120). In addition to volatile storage devices that require power to maintain stored information, the memory (110) can include non-volatile storage devices such as magnetic storage media or flash storage media, but the scope of the present invention is not limited thereto.

The processor (120) can execute the prediction program stored in the memory (110) to predict the binding structure of a protein and a ligand based on received protein information and ligand information, and provide the prediction result.

Referring to FIGS. 2 to 9, the operation of a structure prediction program for predicting a binding structure between a protein and a ligand is described. The structure prediction program receives protein information (10) and ligand information (20), generates a protein vector (30, 40) using the protein information (10), generates a ligand vector (50) using the ligand information (20), and can predict the binding structure between the protein and the ligand based on the protein vector (30, 40) and the ligand information (20).

First, the operation of generating the protein vector (30, 40) using protein information (10) is described. The protein information (10) can be sequence information for a protein composed of multiple residues (11), as shown in FIG. 3. In other words, the protein information (10) represents the sequence structure of residues (11) constituting the protein, from the first residue (11-1) to the nth residue (11-n). In such a case, the residues (11) are one amino acid.

The structure prediction program can generate a first protein vector (30) including evolutionary information for each residue (11) included in the protein information (10) based on a protein database. The structure prediction program can generate the first protein vector (30) by inputting the protein information (10) into a protein-information provision model (111). The protein-information provision model (111) can be trained to learn a protein database to generate a first protein vector by vectorizing evolutionary information for each residue included in the input protein information. The protein-information provision model (111) can also utilize protein language models (PLMs) pre-trained from a large-scale protein database. The protein database can contain experimental information about which residues of a protein bind which ligands. The protein database can contain already known data that can be used for model learning.

The first protein vector (30) is a vectorized version of evolutionary information (31) for each residue (11) constituting the protein, as shown in FIG. 4, and can include evolutionary information (31-1) for the first residue (11-1) to evolutionary information (31-n) for the n-th residue (11-n). The evolutionary information can include at least one of conservation of residues, co-evolutionary information of residues, family-specific sequence patterns of residues, mutation data and pathogenicity prediction of residues, functional site information of residues, and evolutionary homology and comparative data for the amino acid residues, but not limited thereto.

The conservation of a residue refers to the extent to which a particular residue remains the same across species during evolution; the co-evolutionary refers to the tendency of two residues to evolve together, indicating the tendency for other residues to change when on residue mutates; the family-specific sequence patterns can refer to sequence segments or patterns that are common with the same protein family, and the family-specific sequence patterns are used to distinguish the functional classification or characteristics of proteins; the mutation data and pathogenicity prediction refer to the extent to which a mutation in a protein sequence causes a functional defect or disease; the functional site information can refers to the location of residues directly involved in the biological function of a protein; and evolutionary homology and comparative data refer to information that identifies evolutionarily similar sites by comparing protein sequences from different species, but not limited thereto.

In generating the first protein vector, for example, the protein-information provision model (111) can directly generate the first protein vector for each residue in a single step with 1280 dimensions rather than separately calculating and combining the conservation, the co-evolutionary information, the family-specific sequence patterns, the mutation data and pathogenicity prediction, the functional site information, the evolutionary homology, and the comparative data. For instance, if a protein sequence is given as WHQS..., a vectorized representation for each residue, such as, W in 1280 dimensions (containing the evolutionary information thereof), H in 1280 dimensions (containing the evolutionary information thereof), Q in 1280 dimensions (containing the evolutionary information thereof), S in 1280 dimensions (containing the evolutionary information thereof), and the like, can be generated through the protein-information provision model (111).

Further, the structural prediction program can calculate distance information between each residue (11) or calculate coordinate information of each residue (11) in a predetermined space based on the evolutionary information included in the first protein vector (30) and add this information to the first protein vector (30) to generate a second protein vector (40). In other words, structural information for each residue (11) can be generated by adding distance information or coordinate information to the evolutionary information (31) of each residue (11) included in the first protein vector (30).

More specifically, the second protein vector (40) can be generated by inputting the first protein vector (30) into a machine learning model, such as, a BiLSTM model and a 1D-CNN model, for a structural inference. This process allows derivation of new residue-specific vector representations that reflect local patterns and global interaction information in the sequence, even without directly yielding three-dimensional coordinates.

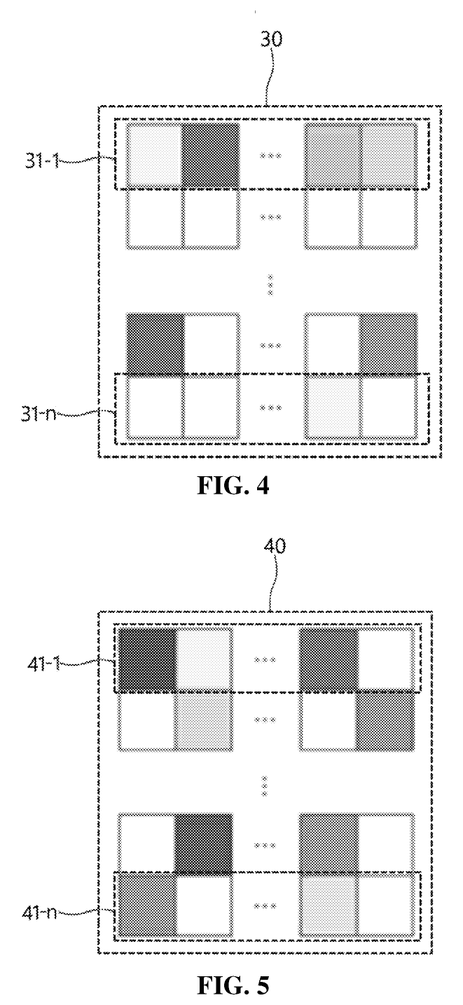

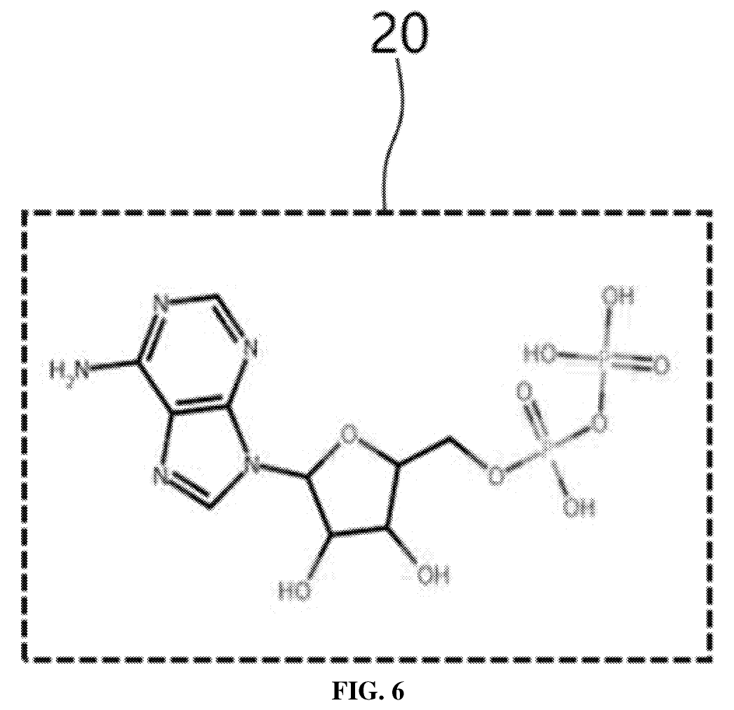

Referring to FIG. 5, the second protein vector (40) includes structural information (41-1) of the first residue (11-1) to structural information (41-n) of the n-th residue (11-n). The structural information (41-1) of the first residue (11-1) can include distance information or coordinate information in addition to the evolutionary information (31-1) of the first protein vector (30).

The structure prediction program can input the first protein vector (30) into a structure inference model (112) so as to generate a second protein vector (40). The structure inference model (112) can be trained to calculate distance information between each residue (11) or coordinate information of each residue (11) for a predetermined space based on the evolutionary information included in the first protein vector (30), and add this to the first protein vector to generate a second protein vector. Further, the structure inference model (112) can be developed by combining a 1D-CNN model and a BiLSTM model. The 1D-CNN model and the BiLSTM model can be combined by passing the output of the 1D-CNN layers, which are used for feature extraction, into the BiLSTM layers for sequential learning, but not limited thereto.

In FIG. 3, the protein information (10) represents sequence information of residues (11), but such sequence information does not include information on the three-dimensional structure of the protein. The first residue (11-1) and the second residue (11-2) are in contact in the sequence, but can be arranged at a predetermined distance apart in the actual three-dimensional structure, and the first residue (11-1) and the n-th residue (11-n) can be far apart in the sequence, however, can be arranged closer than the second residue (11-2) in a three-dimensional structure.

The second protein vector (40) includes such structural information in the evolutionary information for each residue (11), and the present invention can predict the binding site of a protein that binds to a ligand by using the second protein vector (40) including three-dimensional structural information for each residue (11).

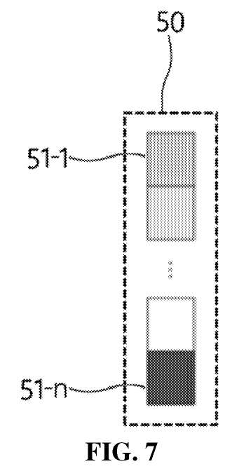

Next, the operation of generating a ligand vector (50) using ligand information (20) is described. The ligand information (20) can be expressed as a chemical structure, as illustrated in FIG. 6. A structure prediction program can generate a ligand vector (50) that vectorizes chemical, physical, and structural information for each atom of the ligand based on the chemical structure of the ligand information (20).

The structure prediction program can input the ligand information (20) into a graph generation model (113), such as, a graph attention network (GAT), to generate the ligand vector (50). The graph generation model (113) can be trained to generate a ligand vector that vectorizes chemical, physical, and structural property information for each atom constituting the ligand based on the ligand information.

Referring to FIG. 7, the ligand vector (50) can include atomic information (51) that includes chemical, physical, and structural information for each atom. The atomic information includes first atomic information (51-1) for the first atom, second atomic information for the second atom, and nth atomic information (51-n) for the nth atom. For example, the chemical, physical, and structural information of each atom can be derived from ligand atom features which include the atom type (such as, C, N, O, F, P, S, Cl, Br, I, other), degree of atom (0, 1, 2, ,3 . . . , 12), formal charge (0 or 1), number of radical electrons (0 or 1), hybridization type (sp, sp2, sp3d, sp3d2, other), aromatic (0 or 1), number of hydrogen atoms attached (0, 1, 2, 3, 4), chirality (0 (false) or 1 (true)), configuration (R or S). Specifically, the ligand atom features are vectorized through a GAT module after being encoded.

Next, the operation of predicting the binding structure of a protein and a ligand based on the second protein vector (40) and the ligand vector (50) is described.

The structure prediction program generates interaction data (60) through operation of the second protein vector (40) and the ligand vector (50), and can estimate the binding structure of a protein bound to a ligand based on the interaction data (60).

Specifically, the operation between the second protein vector (40) and the ligand vector (50) can be obtained by calculation of the element-wise product between the second protein vector (40) and the ligand vector (50). For example, if one of the residues in the protein vector is presented as [1, 2] and the ligand vector is [2, 3], the interaction vector representing the interaction data can be obtained by [1*2, 2*3] = [2, 6]. Thereafter, the generated interaction vector can pass through a fully connected layer and be converted into a probability value for each residue and ligand binding. The probability value can be used for prediction whether the residue and the ligand are binding or non-binding, and the predicted binding site is visualized using a protein 3D visualization tool.

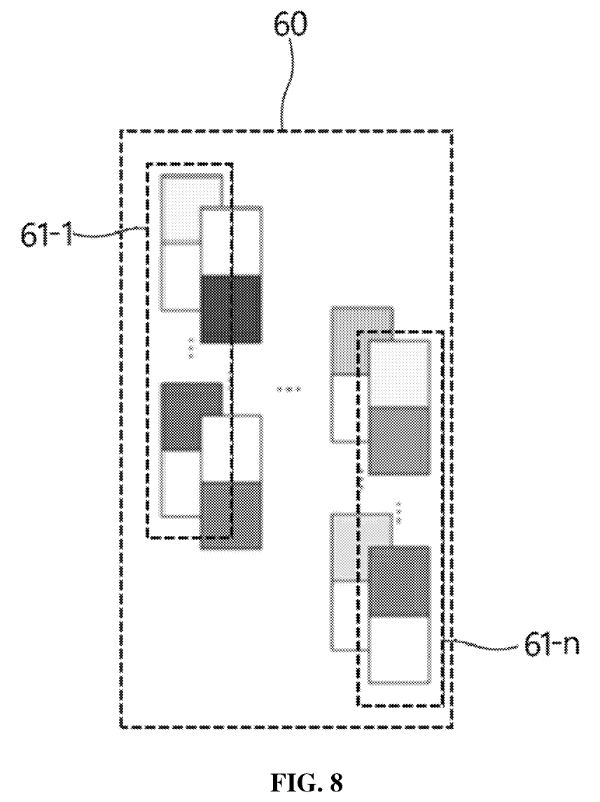

Referring to FIG. 8, the interaction data (60) is obtained by calculating the second protein vector (40) illustrated in FIG. 5 and the ligand vector (50) illustrated in FIG. 7, and the interaction data (60) includes interaction information between each residue and the ligand vector (50). First interaction information (61-1) is information obtained by calculating the first structural information (41-1) for the first residue and the ligand vector (50), representing interaction information between the first residue (11-1) and the ligand, and n-th interaction information (61-n) is information obtained by calculating the n-th structural information (41-n) for the n-th residue and the ligand vector (50), representing interaction information between the n-th residue (11-n) and the ligand.

In this regard, a structure prediction program can input such interaction data (60) into a binding prediction model (114) and output a binding structure (70) of a protein and a ligand based on the interaction data (60). The binding prediction model (114) is trained to calculate the degree of binding between each of the residues and the ligand based on the interaction data, and determine whether each residue binds to the ligand based on the degree of binding. Here, the binding structure (70) includes a plurality of binding information indicating whether each residue binds to the ligand.

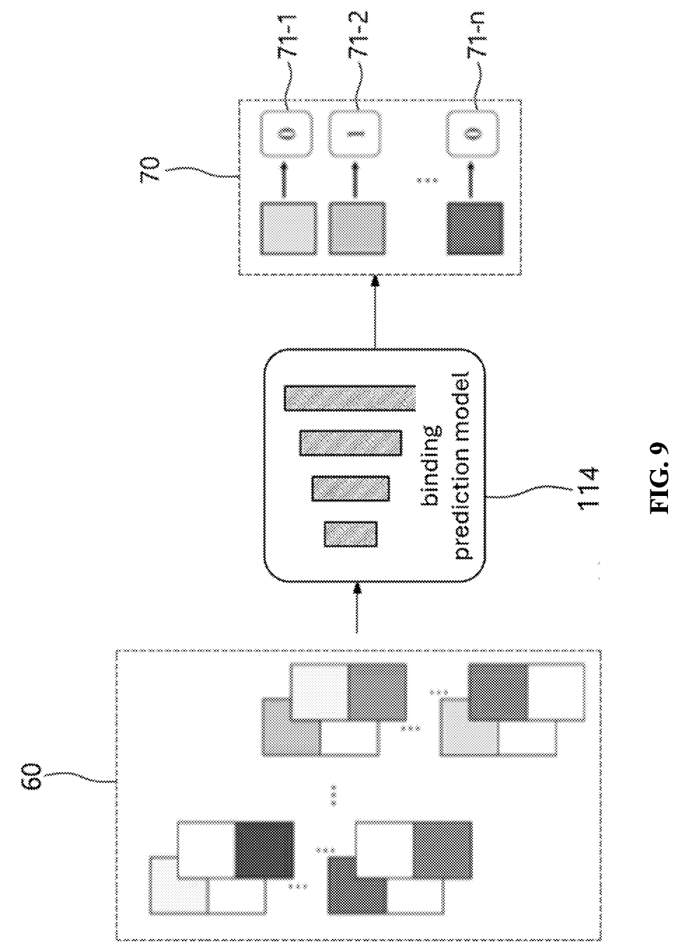

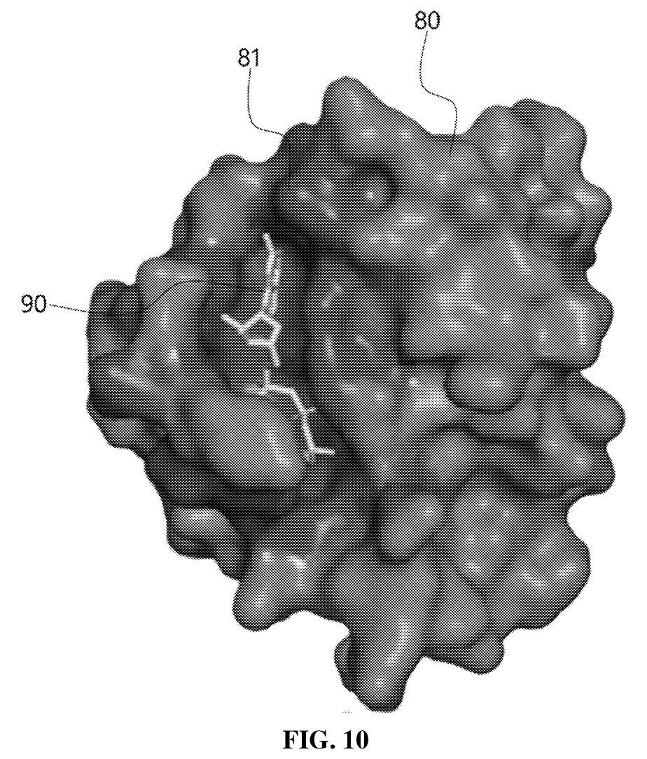

Referring to FIG. 9, a structure prediction program inputs the interaction data (60) into a binding prediction model (114), and the binding prediction model (114) can output a binding structure (70) between a ligand and a protein based on the interaction data (60). The binding structure (70) includes multiple binding information (71) indicating whether the ligand binds to each residue. The binding information (71) is based on the binding strength calculated using the interaction information between each residue and the ligand. When the binding strength is greater than a threshold value, the residue and the ligand are determined to be bound, and is represented as 1. When the binding strength is less than the threshold value, the residue and the ligand are determined not to be bound, and is represented as 0.

In FIG. 9, as the binding information (71-1) between the first residue and the ligand is presented as 0, the first residue (71-1) and the ligand are not bound to each other, and as the binding information (71-2) between the second residue and the ligand is presented as 1, the second residue (71-2) is bound to the ligand.

Further, referring to FIG. 10, the structure prediction program can generate a three-dimensional structure image (80) of a protein based on the second protein vector (40), and provide a binding site (81) of a protein (80) that binds to a ligand (90) by expressing it in the structure image (80) based on the binding result (70), the binding result (70) being an output value of the binding prediction model (114).

Meanwhile, the processor (120) can perform hardware control functions, such as, file systems, memory allocation, networks, basic libraries, timers, device control (display, media, input devices, 3D, or the like), and other utilities, as necessary for executing a program. In the present embodiment, the processor (120) can be implemented in the form of a microprocessor, a central processing unit (CPU), a processor core, a multiprocessor, an application-specific integrated circuit (ASIC), a field programmable gate array (FPGA), or the like, but the scope of the present invention is not limited thereto.

The communication module (130) encompasses a device that includes hardware and software required to transmit and receive signals, such as, control signals or data signals, with other network devices via wired or wireless connections to perform data communication with external devices. The database (140) can store various data required for the operation of the structure prediction program. For example, data required for the operation of a structure prediction program, such as, a protein-information provision model (111), a structure inference model (112), a graph generation model (113), and a binding prediction model (114), can be stored.

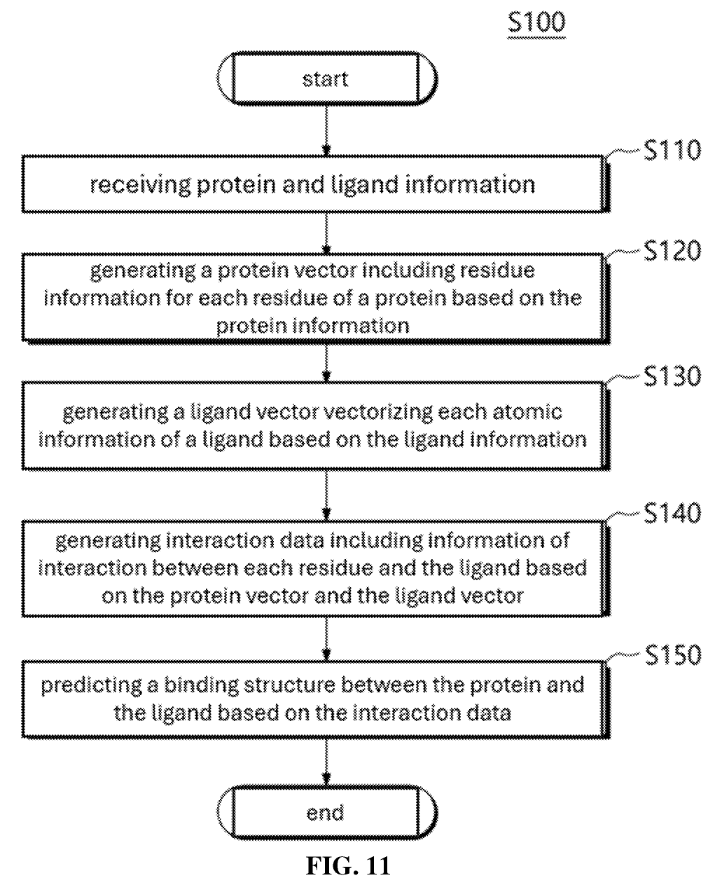

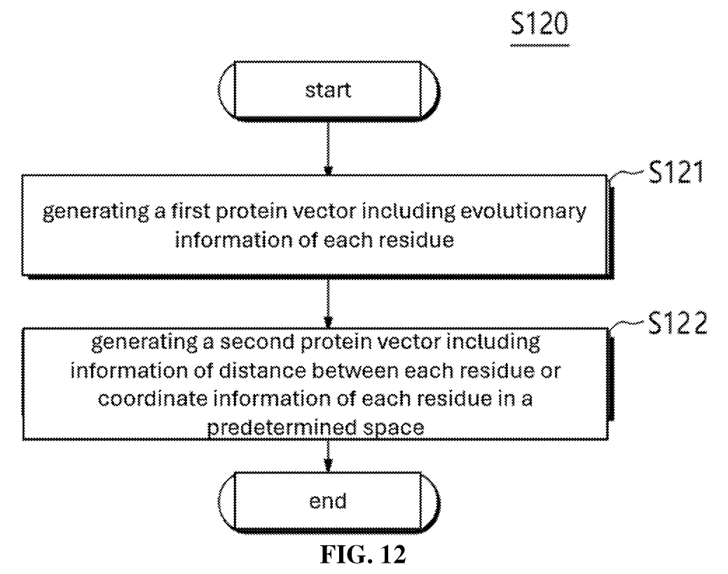

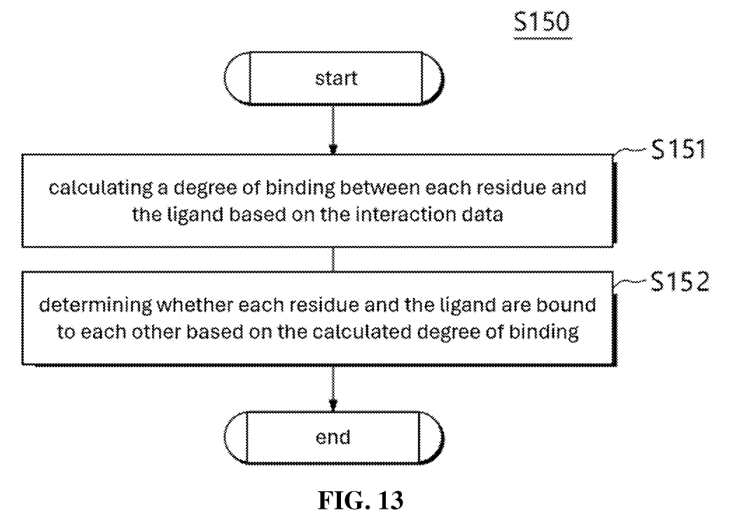

FIG. 11 is a flowchart explaining a method for predicting a protein-ligand binding structure according to an embodiment of the present invention, FIG. 12 is a flowchart explaining a process for generating a protein vector illustrated in FIG. 11, and FIG. 13 is a flowchart explaining a process for predicting a binding structure between a ligand and a protein illustrated in FIG. 11.

Referring to FIGS. 1, 2, and 11, a protein-ligand binding structure prediction method using a protein-ligand binding structure prediction apparatus (100) is described. The protein-ligand binding structure prediction method (S100) can be configured such that the protein-ligand binding structure prediction apparatus (100) receives protein information (10) and ligand information (20), and generates a protein vector (30, 40) including residue information for each residue constituting the protein based on the protein information (10) (step S120). Further, based on the ligand information (20), a ligand vector (50) that vectorizes each atomic information of the ligand can be generated (step S130), and interaction data (60) including interaction information between the protein and the ligand can be generated (step S140).

Furthermore, based on the interaction data (60), the residues of the protein that bind to the ligand can be estimated, and thus, the binding structure (70) between the protein and the ligand can be predicted (step S150).

Next, in reference to FIGS. 2 to 10, each step of the protein-ligand binding structure prediction method (S100) is described in detail.

First, a process (step S120) for generating a protein vector (30, 40) using protein information (10) is described. The protein information (10) can be sequence information for a protein including multiple residues (11), as shown in FIG. 3. In other words, the protein information presents the sequence structure of residues (11) constituting the protein, from the first residue (11-1) to the nth residue (11-n). In this case, the residues (11) can represent one amino acid.

The protein ligand binding structure prediction apparatus (100) can generate a first protein vector (30) including evolutionary information for each residue (11) constituting the protein based on the protein information (10) (step S121), and can generate a second protein vector (40) including distance information for each residue or coordinate information for each residue in a predetermined space based on the first protein vector (30) (step S122).

Specifically describing the process of generating the first protein vector (30) (step S121), the protein ligand binding structure prediction apparatus (100) can generate the first protein vector (30) including evolutionary information for each residue (11) included in the protein information (10) based on a protein database (step S121). At the moment, the protein ligand binding structure prediction apparatus (100) can generate the first protein vector (30) by inputting the protein information (10) into a protein-information provision model (111). The protein-information provision model (111) can be trained to learn a protein database to generate the first protein vector that vectorizes the evolutionary information for each residue included in the input protein information.

The first protein vector (30) can represent vectorized features of evolutionary information (31) for each residue (11) constituting the protein, as illustrated in FIG. 4. The first protein vector (30) includes evolutionary information (31-1) for the first residue (11-1) to evolutionary information (31-n) for the nth residue (11-n). The evolutionary information can include one or more of conservation, co-evolutionary information, family-specific sequence patterns, mutation data and pathogenicity prediction, functional site information, and evolutionary homology and comparative data for the amino acid residues.

Further, specifically describing the process of generating the second protein vector (40) (step S122), the protein ligand binding structure prediction apparatus (100) can calculate distance information between each residue (11) or coordinate information of each residue (11) in a predetermined space based on the evolutionary information included in the first protein vector (30), and add the distance information or the coordinate information to the first protein vector (30) so as to generate the second protein vector (40). In other words, structural information for each residue (11) can be generated by adding the distance information or the coordinate information to the evolutionary information (31) of each residue (11) included in the first protein vector (30). How the structural information for each residue is obtained can be explained in light of FIG. 5. Referring to FIG. 5, the second protein vector (40) can include structural information (41-1) of the first residue (11-1) to structural information (41-n) of the n-th residue (11-n). The structural information (41-1) for the first residue (11-1) can include distance information and/or coordinate information in addition to the evolutionary information (31-1) of the first protein vector (30). In this regard, the protein ligand binding structure prediction device (100) can input the first protein vector (30) into the structure inference model (112) to generate a second protein vector (40). The structure inference model (112) can be trained to calculate distance information between each residue (10) or coordinate information of each residue (11) for a predetermined space based on evolutionary information included in the first protein vector (30), and add the distance information and/or the coordinate information to the first protein vector to generate a second protein vector.

Next, a process (step S130) for generating a ligand vector (50) using ligand information (20) is described. The ligand information (20) can be received as a chemical structure or simplified molecular input line entry system (SMILES) as shown in FIG. 6. A structure prediction program can generate a ligand vector (50) that vectorizes chemical, physical, and structural information for each atom of the ligand based on the chemical structure of the ligand information (20).

In this regard, the protein ligand binding structure prediction apparatus (100) can input ligand information (20) into a graph generation model (113) to generate a ligand vector (50). The graph generation model (113) can be trained to generate a ligand vector that vectorizes chemical, physical, and structural characteristic information of each atom constituting the ligand based on the ligand information.

Referring to FIG. 7, the ligand vector (50) encompasses atomic information (51) that includes chemical, physical, and structural information for each atom. The atomic information can include first atomic information (51-1) for the first atom to nth atomic information (51-n) for the nth atom.

Next, the process of generating interaction data (60) based on the protein vectors (30, 40) and the ligand vector (50) (step S140) is described. The protein-ligand binding structure prediction apparatus (100) can generate interaction data (60) through calculations or operations between the second protein vector (40) and the ligand vector (50).

Referring to FIG. 8, the interaction data (60) is obtained by calculating or operating between the second protein vector (40) of FIG. 5 and the ligand vector (50) of FIG. 7, and can include interaction information between each residue and the ligand vector (50). The first interaction information (61-1) is information obtained by calculating or operating the first structural information (41-1) of the first residue and the ligand vector (50), and can represent interaction information between the first residue (11-1) and the ligand, and the n-th interaction information (61-n) is information obtained by calculating the n-th structural information (41-n) for the n-th residue and the ligand vector (50), and can represent interaction information between the n-th residue (11-n) and the ligand.

Next, referring to FIG. 13, a process (step S150) for predicting a binding structure (70) of a ligand and a protein based on interaction data (60) is described. A protein-ligand binding structure prediction apparatus (100) can estimate a binding structure of the protein capable of being bound to the ligand based on the interaction data (60). More specifically, the protein-ligand binding structure prediction apparatus (100) can calculate a binding degree between each residue and the ligand based on the interaction data (60) (step S151), and determine whether each residue binds to the ligand based on the binding degree, thereby providing a binding structure (70) including binding information indicating whether each residue binds to the ligand (step S152).

In the process of predicting the binding structure (70) of the ligand and the protein based on the interaction data (60) (step S150), the protein-ligand binding structure prediction apparatus (100) can input the interaction data (60) into a binding prediction model (114) to output the binding structure (70) of the protein and the ligand. The binding prediction model (114) can be trained to calculate the binding degree between each residue and the ligand based on the interaction data, and determine whether each residue is bound to the ligand based on the binding degree. In this regard, the binding structure (70) can include binding information indicating whether each residue is bound to the ligand.

Referring to FIG. 9, a protein-ligand binding structure prediction apparatus (100) inputs interaction data (60) into a binding prediction model (114), and the binding prediction model (114) can output a binding structure (70) between a ligand and a protein based on the interaction data (60). The binding structure (70) includes multiple binding information (71) indicating whether the ligand binds to each residue. The binding information (71) is based on a binding degree calculated based on interaction information between each residue and the ligand. If the binding degree is greater than a threshold value, the residue and the ligand are determined to be bound, and thus, is represented as 1. If the binding degree is less than the threshold value, the residue and the ligand are determined not to be bound, and thus, is represented as 0.

In FIG. 9, since the binding information (71-1) between the first residue and the ligand is expressed as 0, the first residue (71-1) is presented to be not bound to the ligand, and since the binding information (71-2) between the second residue and the ligand is expressed as 1, the second residue (71-2) is presented to be bound to the ligand.

For instance, a prediction of a binding structure (70) between a protein having VPMTSGAQC and a ligand 'Nc1nc' represented in SMILES can yield V-0, P-1, M-0, T-1, S-1, G-1, A-1, Q-0, C-0. In this example, each of the residues predicted to be bound to the ligand has a value '1', and the rest of the residues predicted to be not bound to the ligand will have a value '0'. The residues constituting the protein can be provided based on the sequence order.

Further, referring to FIG. 10, a protein-ligand binding structure prediction apparatus (100) can generate a three-dimensional structure image (80) of a protein based on a second protein vector (40), and provide a binding site (81) of a protein (80) that binds to a ligand (90) by expressing it in a structure image (80) based on a binding result (70), the binding result (70) being an output value of the binding prediction model (114).

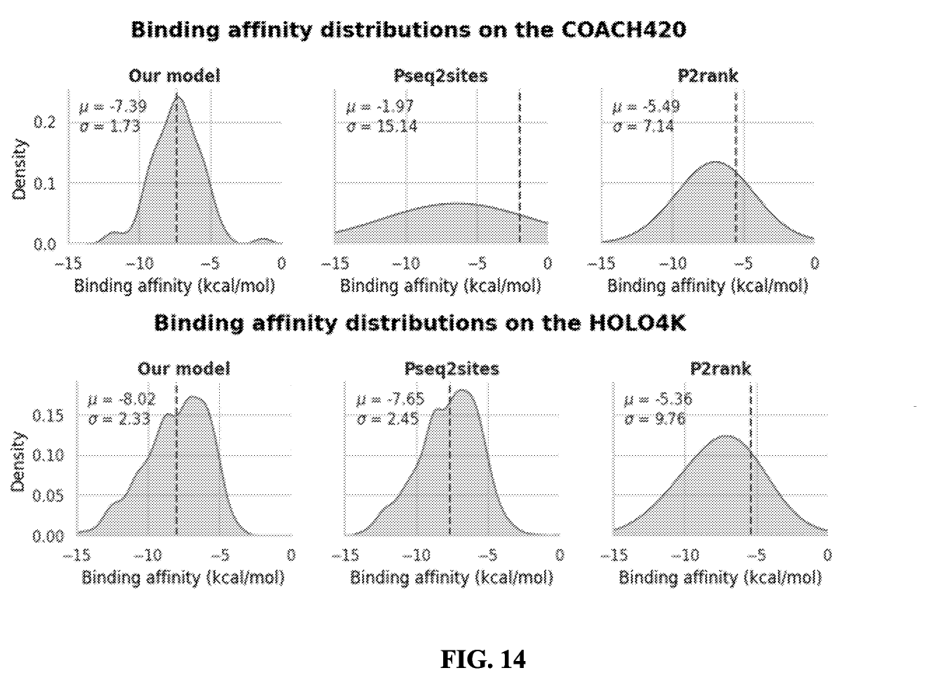

FIG. 14 illustrates experimental results comparing the binding affinities predicted by each prediction model for each test set (COACH420 and HOLO4K). The prediction models used in the experiment are the protein-ligand binding structure apparatus (100) according to the present invention, Pseq2sites (conventional model), and P2rank (conventional model). The test sets COACH420 and HOLO4K are respectively datasets composed of protein-ligand complexes used to evaluate models for predicting protein-ligand binding sites and the same binding sites are measured by each model.

With respect to the binding affinities, the more similarly the predicted binding residues are positioned to the actual binding site, the more stable the ligand binding is, resulting in a lower binding affinity value (which means strong binding). Conversely, if the predicted binding site does not match the actual binding site or if the predicted binding site is more distant from the actual binding site, ligand binding will be unstable, resulting in a relatively high binding affinity value (which means weak binding).

Here, each prediction model measured the binding affinities of the datasets of protein-ligand complexes (COACH420 and HOLO4K), each of the datasets having binding sites of the protein bound to the ligand. As shown in FIG. 14, for COACH420, the binding affinity for the prediction apparatus according to the present invention shows around -7.5, the binding affinity for Pseq2sites shows around -2, and the binding affinity for P2rank shows around -5.5. For HOLO4K, the binding affinity for the prediction apparatus according to the present invention shows around -8, the binding affinity for Pseq2sites shows around -7.5, and the binding affinity for P2rank shows around -5.5. As stated above, the test sets of COACH420 and HOLO4K are the datasets of protein-ligand complexes, meaning each test set has actual binding sites, and therefore, it can be shown that the model showing the lowest binding affinity (meaning the strongest binding) predicts the most structurally accurate binding site among the prediction models. In other words, the lowest binding affinity is interpreted as evidence that the model has captured the structurally accurate binding site. This experiment confirms that the accuracy of the protein-ligand binding structure apparatus (100) has been improved compared to the conventional models.

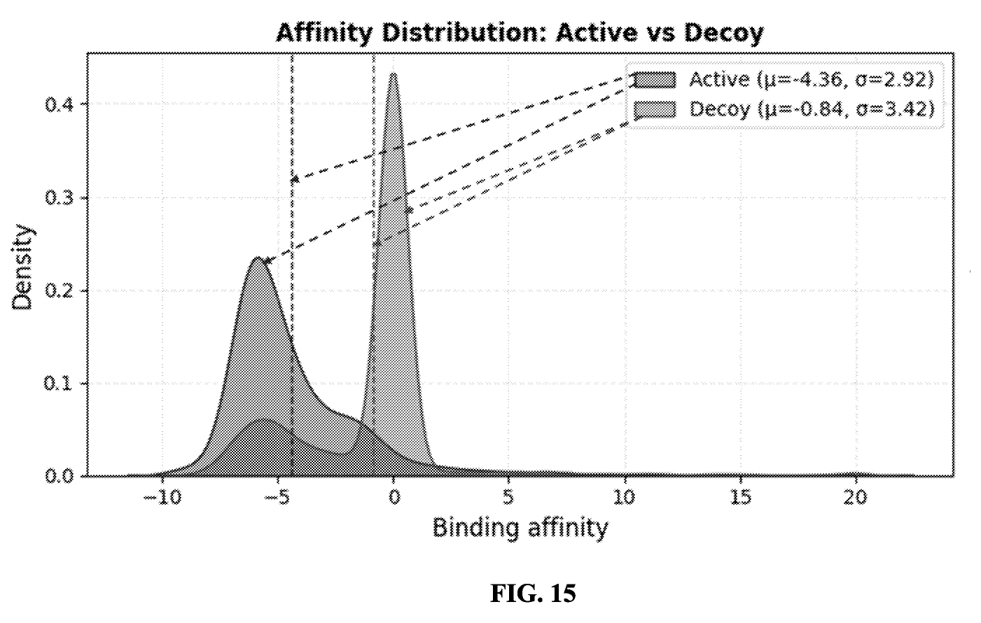

Further, another experiment has been conducted to see the accuracy of the protein-ligand binding structure apparatus (100) according to the present invention. In this experiment, datasets of a particular protein and two ligands, including one (active ligand) of which is actually bound to the protein and the other (decoy ligand) is not bound to the protein, are input to the protein-ligand binding structure apparatus (100). As illustrated in FIG. 15, the binding affinity of the active ligand (around -4.5) shows lower value than the binding affinity of the decoy ligand (around -0.5), which demonstrates that the prediction apparatus (100) precisely captures the binding site and distinguish ligands that are bound to the protein (active ligands) from ligands that are not bound to the protein (decoy ligands). This experiment also manifests that the protein-ligand binding structure apparatus (100) has achieved an improvement in terms of prediction accuracy.

The above description is merely an example of the technical idea of the present embodiment, and those skilled in the art will appreciate that various modifications and variations can be made without departing from the essential characteristics of the present embodiment. Therefore, the present embodiments are not intended to limit the technical idea of the present embodiment, but rather to explain it, and the scope of the technical idea of the present embodiment is not limited by these embodiments. The scope of protection of the present embodiment should be interpreted based on the claims below, and all technical ideas within a scope equivalent thereto should be interpreted as being included in the scope of rights of the present embodiment.

1. An apparatus for predicting a binding structure between a protein including residues and a ligand including atoms, the apparatus comprising:
at least one memory storing a computer program code for predicting a structure of the protein; and
at least one processor configured to execute the computer program code,
wherein the computer program code, when executed by the at least one processor, is configured, with the at least one processor, to cause the apparatus at least to:
receive protein information and ligand information;
generate, based on the protein information, a protein vector including information of a distance between each residue of the protein;
generate, based on the ligand information, a ligand vector by vectorizing information of the atoms of the ligand;
generate interaction data including interaction information between each residue of the protein and the ligand based on the protein vector and the ligand vector; and
predict the structure based on the interaction data.

2. The apparatus of claim 1,
wherein the computer program code is configured to predict each residue binding to the ligand based on the interaction data.

3. The apparatus of claim 2,
wherein the computer program code is configured to generate the ligand vector including chemical and physical property information of the atoms of the ligand by inputting the ligand information to a graph generation model, and
wherein the graph generation model is trained to vectorize the chemical and physical information of the atoms of the ligand based on the ligand information.

4. The apparatus of claim 1,
wherein the computer program code is configured to generate a first protein vector by vectorizing evolutionary information for each residue of the protein based on a protein database.

5. The apparatus of claim 4,
wherein the computer program code is configured to generate the first protein vector by inputting the protein information to a protein information provision model, and
wherein the protein information provision model is trained to generate the first protein vector based on the protein information.

6. The apparatus of claim 4,
wherein the computer program code is configured to generate, based on the first protein vector, a second protein vector including the information of the distance between each residue.

7. The apparatus of claim 6,
wherein the computer program code is configured to generate the second protein vector by inputting the first protein vector into a structure inference model, and
wherein the structure inference model is trained to:
calculate the distance between each residue based on the evolutionary information included in the first protein vector; and
generate the second protein vector by adding the information the distance between each residue to the first protein vector.

8. The apparatus of claim 6,
wherein the computer program code is configured to generate, based on the second protein vector and the ligand vector, the interaction data including the interaction information between each residue and the ligand.

9. The apparatus of claim 8,
wherein the computer program code is configured to input the interaction data to a binding prediction model so as to identify one or more of the residues which bind to the ligand, and
wherein the binding prediction model is trained to:
calculate a degree of binding between each residue and the ligand based on the interaction data; and
determine whether each residue and the ligand are bound to each other based on the calculated degree of binding.

10. An apparatus for predicting a binding structure between a protein including residues and a ligand, the apparatus comprising:
at least one memory storing a computer program code for predicting a structure of the protein capable of being bound to the ligand; and
at least one processor configured to execute the computer program code,
wherein the computer program code, when executed by the at least one processor, is configured, with the at least one processor, to cause the apparatus at least to:
receive protein information and ligand information;
generate, based on the protein information, a protein vector including coordinate information of each residue of the protein;
generate, based on the ligand information, a ligand vector including chemical and physical property information of the ligand;
generate, based on the protein vector and the ligand vector, interaction data including interaction information between each residue and the ligand; and
predict the structure based on the interaction data.

11. The apparatus of claim 10,
wherein the computer program code is configured to generate a first protein vector by vectorizing evolutionary information for each residue of the protein based on a protein database.

12. The apparatus of claim 11,
wherein the computer program code is configured to generate, based on the first protein vector, a second protein vector including coordinate information of each residue.

13. The apparatus of claim 12,
wherein the computer program code is configured to generate the second protein vector by inputting the first protein vector into a structure inference model, and
wherein the structure inference model is trained to:
calculate the coordinate information of each residue in the predetermined space based on the evolutionary information for each residue included in the first protein vector; and
generate the second protein vector by adding the coordinate information of each residue to the first protein vector.

14. A method for predicting a binding structure between a protein including residues and a ligand including atoms using a binding structure prediction apparatus, the method comprising:
receiving protein information and ligand information;
generating, based on the protein information, a protein vector including information of a distance between each residue of the protein;
generating, based on the ligand information, a ligand vector by vectorizing information of the atoms of the ligand;
generating, based on the protein vector and the ligand vector, interaction data including interaction information between each residue and the ligand; and
predicting the binding structure based on the interaction data.

15. The method of claim 14,
wherein the generating of the protein vector includes:
generating a first protein vector by vectorizing evolutionary information for each residue of the protein based on a protein database; and
generating, based on the first protein vector, a second protein vector including the information of the distance between each residue.

16. The method of claim 14,
wherein the predicting of the structure includes:
calculating a degree of binding between each residue and the ligand based on the interaction data; and
determining whether each residue and the ligand are bound to each other based on the calculated degree of binding.

17. A method for predicting a binding structure between a protein including residues and a ligand including atoms using a binding structure prediction apparatus, the method comprising:
receiving protein information and ligand information;
generating, based on the protein information, a protein vector including coordinate information of each residue of the protein;
generating, based on the ligand information, a ligand vector by vectorizing information of the atoms of the ligand;
generating, based on the protein vector and the ligand vector, interaction data including interaction information between each residue and the ligand; and
predicting the binding structure between the protein and the ligand based on the interaction data.

18. The method of claim 17,
wherein the generating of the protein vector includes:
generating, based on a protein database, a first protein vector by vectorizing evolutionary information for each residue of the protein; and
generating, based on the first protein vector, a second protein vector including the coordinate information of each residue.

19. A non-transitory computer-readable storage medium storing a program configured to perform the method of claim 14.

20. A non-transitory computer-readable storage medium storing a program configured to perform the method of claim 17.
